(19) United States
(12) Patent Application Publication
(10) Pub. No.: US 2025/0382262 A1
(43) Pub. Date: Dec. 18, 2025

(54) PROCESS FOR THE PREPARATION OF BIGUANIDINE SALTS AND TRIAZINES

(71) Applicant: ADAMA AGAN LTD., Ashdod (IL)

(72) Inventors: Einat KISIN-FINFER, Hod Hasharon (IL); Reut FALLEK, Ashdod (IL); Ira AGRANOVICH, Ashdod (IL)

(73) Assignee: ADAMA AGAN LTD., Ashdod (IL)

(21) Appl. No.: 18/879,151

(22) PCT Filed: Jun. 29, 2023
(86) PCT No.: PCT/IL2023/050675

§ 371 Date: Dec. 26, 2024

(30) Foreign Application Priority Data
Jun. 30, 2022 (EP) 22182294.3

(51) Int. Cl. C07C 277/08; C07D 251/18

(52) U.S. Cl. CPC C07C 277/08; C07D 251/18; C07C 2602/08

(57) ABSTRACT

A process for the preparation of biguanidine compounds of formula (I) and triazine compounds of formula (IV); wherein R1, R2 and R3 are each independently hydrogen or an optionally substituted C1-C4 alkyl group, wherein the optional substituents are selected from the group consisting of halogen, aliphatic, halo aliphatic, alicyclic, alkoxy, thioalkyl, cyano or nitro groups; A is —CH2—, —O— or a direct bond; and n is 0, 1, 2 or 3.

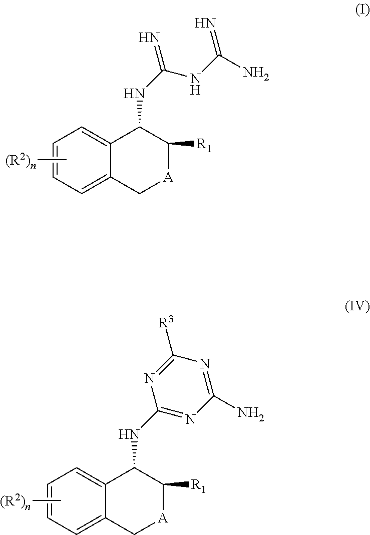

### FIELD OF THE INVENTION

The present invention belongs to the field of agrochemistry. It is directed to a process for preparation of biguanidines and triazines, wherein the triazines are suitable as herbicides for controlling weeds.

### BACKGROUND

Triazine compounds are a class of compounds suitable for being used as herbicides. Triazine compounds such as atrazine, ametryne, indaziflam or triaziflam are among the compounds that are used as herbicides

The prior art discloses in EP1592674 A1, EP2231679 A1 or EP3347342 A1 different methods for preparing indaziflam. One of the methods disclosed in the prior art document EP1592674 A1 relates to the preparation of biguanidine intermediates of formula (I):

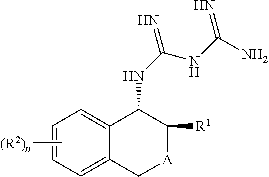

from the reaction of 1-cyanoguanidine with an amine of formula (II):

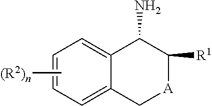

in a solvent such as 1,2-dichlorobenzene, decalin or white mineral oil, at a temperature of from 20° C. to the reflux temperature of the solvent, preferably at 50° C. to 200° C.

According to that document, the biguanidine intermediate of formula (I) is obtained with a yield of 67.7% when the reaction of (1R,2S)-1-amino-2-methylindane hydrochloride and 1-cyanoguanidine is performed in 1,3-dichlorobenzene at 140-150° C.

Also according to this document EP1592674 A1, the biguanidine intermediate of formula (I) or an acid addition salt thereof reacts with a carboxylic acid derivative of formula (III): Z—R³, in the presence of a base, in an inert solvent such as e.g. a polar organic solvent such as tetrahydrofuran, dioxan, acetonitrile, N,N-dimethylformamide, methanol or ethanol, at a temperature of from 0° C. to the reflux temperature of the solvent, preferably at 20° C. to 100° C., to yield a triazine compound of formula (IV):

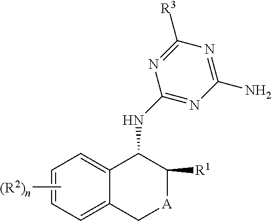

This synthesis of compounds of formula (IV) according to the herein disclosed synthesis in EP1592674 A1 shows the drawback of the low yield in the preparation of biguanidine intermediate of formula (I) or an acid addition salt thereof.

Additional approach described in the prior art is described in EP2231679 A1. That document refers to the use of aluminum alkoxides as an additive in a first step to form a biguanidino-aluminum complex. The addition of an aluminum alkoxide allows the first reaction to progress under low temperature and form a stable biguanidino-aluminum complex intermediate which also reacts in a “one-pot” fashion to afford the final indaziflam with better yield than the yield reported in EP1592674 A1. However, the approach in this prior art document requires the use of large excess of aluminum alkoxide, which results later on in considerable amounts of waste which requires costly and complex disposal and it is a major drawback in industrial production. The process also requires a combination of several different solvents for each step, which is an operational challenge while trying to recycle and isolate each of them.

The third method for preparing indaziflam know in the prior art is disclosed in EP3347342 A1. According to that document, the hereinabove disclosed preparation of indaziflam according to EP1592674 A1 is achieved with higher yield than in EP1592674 A1 when an autocatalytic amount of the biguanidine intermediate of formula (I) is added to the preparation of the biguanidine intermediate of formula (I) and the reaction is preferably performed at a temperature from 140° C. to 148° C. The process also offers a “one-pot” synthesis, while the second step from the biguanidine intermediate of formula (I) to indaziflam involves the addition of a phase transfer catalyst and potassium carbonate as a base. However, the first step is still performed under very high temperatures and the process also requires the filtration of the excess of potassium carbonate salt, which makes the isolation more complicated operationally.

Therefore, the present invention provides an alternative to the existing methods for the preparation of indaziflam and it is an object of the present invention to provide a process which solves some of the drawbacks of low yield, considerable amounts of waste to be handled, high temperature of the reaction or operationally complicated isolation.

### SUMMARY OF THE INVENTION

A first aspect of the invention is a process for the preparation of a biguanidine compound of formula (I) or an acid addition salt thereof:

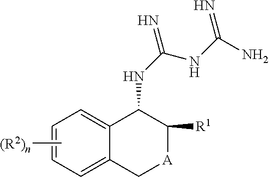

wherein R¹ and R² are each independently hydrogen or an optionally substituted C₁-C₄ alkyl group, wherein the optional substituents are selected from the group consisting of halogen, aliphatic, halo aliphatic, alicyclic, alkoxy, thioalkyl, cyano or nitro groups; A is —CH₂—, —O— or a direct bond; and n is 0, 1, 2 or 3;
which comprises the reaction of 1-cyanoguanidine with an amine of formula (II) or an acid addition salt thereof

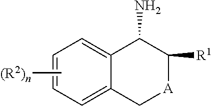

wherein R¹, R², A and n are as defined in formula (I), in a mixture of a polar aprotic solvent with a dielectric constant higher than 12.0, when measured at 25° C., and a water immiscible solvent.

A second aspect of the invention is a process for the preparation of a triazine compound of formula (IV):

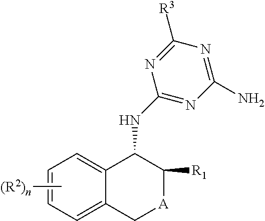

wherein R¹, R² and R³ are each independently hydrogen or an optionally substituted C₁-C₄ alkyl group, wherein the optional substituents are selected from the group consisting of halogen, aliphatic, halo aliphatic, alicyclic, alkoxy, thioalkyl, cyano or nitro groups; A is —CH₂—, —O— or a direct bond; and n is 0, 1, 2 or 3; which comprises
a) a first step of preparation of a biguanidine compound of formula (I) or an acid addition salt thereof:

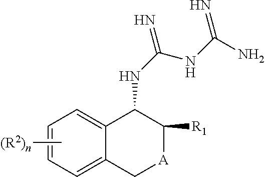

wherein R¹, R², A and n are as defined in formula (IV);
by reaction of 1-cyanoguanidine with an amine of formula (II) or an acid addition salt thereof

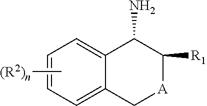

wherein R¹, R², A and n are as defined in formula (IV), in a mixture of a polar aprotic solvent with a dielectric constant higher than 12.0, when measured at 25° C., and a water immiscible solvent;
b) a base and a carboxylic acid derivative of formula (III) are added to the biguanidine compound of formula (I) or an acid addition salt thereof, obtained in step a)

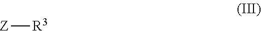

wherein R³ is as defined in formula (IV), and Z—R³ is selected from the group consisting of carboxylic acid esters, carboxylic orthoesters, carboxylic acid chlorides, carboxamides, nitriles, or carboxylic anhydrides

### DETAILED DESCRIPTION OF THE INVENTION

### Definitions

Embodiments of the present invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. While a number of embodiments and features are described herein, it is to be understood that the various features of the invention and aspects of embodiments, even if described separately, may be combined unless mutually exclusive or contrary to the specific description. All references cited herein are incorporated by reference as if each had been individually incorporated.

As used herein, the transitional term “comprising” or “that comprises”, which is synonymous with “including,” or “containing,” is inclusive or open-ended and does not exclude additional, un-recited elements or method steps. However, in each recitation of “comprising” herein, it is intended that the term also encompass, as alternative embodiments, the phrases “consisting essentially of” and “consisting of”, where “consisting of” excludes any element or step not specified and “consisting essentially of” permits the inclusion of additional un-recited elements or steps that do not materially affect the essential or basic and novel characteristics of the composition or method under consideration.

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

The term “alkoxy group” refers to an alkyl group bound to oxygen.

The term “thioalkyl group” refers to an alkyl group bound to sulfur.

The term “halo aliphatic group” refers to an aliphatic group substituted with at least one halogen atom.

The term “aliphatic group” is used in this invention to cover, for example and not restricted to, the linear or branched alkyl, alkenyl and alkynyl groups.

The term “alkyl group” refers to a saturated, linear or branched group, which has between 1 and 24, between 1 and 16, between 1 and 14, between 1 and 12, 1, 2, 3, 4, 5 or 6 carbon atoms and is bound to the rest of the molecule by a single bond, including, for example and not restricted to, methyl, ethyl, isopropyl, isobutyl, tert-butyl, heptyl, octyl, decyl, dodecyl, hexadecyl, octadecyl, amyl, 2-ethylhexyl, 2-methylbutyl, 5-methylhexyl and similar.

The term “alkenyl group” refers to a linear or branched group, which has between 2 and 24, between 2 and 16, between 2 and 14, between 2 and 12, 2, 3, 4, 5 or 6 carbon atoms, with 1, 2 or 3 carbon-carbon double bonds, conjugated or unconjugated, which is bound to the rest of the molecule by a single bond, including, for example and not restricted to, vinyl, allyl, oleyl, linoleyl and similar groups.

The term “alkynyl group” refers to a linear or branched group, which has between 2 and 24, between 2 and 16, between 2 and 14, between 2 and 12, 2, 3, 4, 5 or 6 carbon atoms, with 1, 2 or 3 carbon-carbon triple bonds, conjugated or unconjugated, which is bound to the rest of the molecule by a single bond, including, for example and not restricted to, the ethynyl group, 1-propinyl, 2-propinyl, 1-butinyl, 2-butinyl, 3-butinyl, pentinyl, such as 1-pentinyl, and similar.

The term “alkylidene group” refers to a saturated, linear or branched group, which has between 1 and 24, between 1 and 16, between 1 and 14, between 1 and 12, 1, 2, 3, 4, 5 or 6 carbon atoms and is bound to the rest of the molecule by a double bond, including, for example and not restricted to, methylidene, ethylidene, isopropylidene, isobutylidene, tert-butylidene, heptylidene, octylidene, decylidene, dodecylidene, hexadecylidene and similar.

The term “alycyclic group” is used in this invention to cover, for example and not restricted to, cycloalkyl or cycloalkenyl or cycloalkynyl groups.

The term “cycloalkyl” refers to a saturated mono- or polycyclic aliphatic group which has between 3 and 24, between 3 and 16, between 3 and 14, between 3 and 12, between 3, 4, 5 or 6 carbon atoms and which is bound to the rest of the molecule by a single bond, including, for example and not restricted to, cyclopropyl, cyclobutyl, cyclopentyl, cyclohexyl, cycloheptyl, methyl cyclohexyl, dimethyl cyclohexyl, octahydroindene, decahydronaphthalene, dodecahydrophenalene and similar.

The term “cycloalkenyl” refers to a non-aromatic mono- or polycyclic aliphatic group which has between 5 and 24, between 5 and 16, between 5 and 14, between 5 and 12, 5 or 6 carbon atoms, with 1, 2 or 3 carbon-carbon double bonds, conjugated or unconjugated, and which is bound to the rest of the molecule by a single bond, including, for example and not restricted to, the cyclopent-1-en-1-yl group and similar.

The term “cycloalkynyl” refers to a non-aromatic mono- or polycyclic aliphatic group which has between 8 and 24, between 8 and 16, between 8 and 14, between 8 and 12, 8 or 9 carbon atoms, with 1, 2 or 3 carbon-carbon triple bonds, conjugated or unconjugated, and which is bound to the rest of the molecule by a single bond, including, for example and not restricted to, the cyclooct-2-in-1-yl group and similar.

As used herein, the term “water immiscible solvent” refers to a solvent that is not water miscible, or hardly miscible, and in particular, the term “water immiscible solvent” is used in this invention to cover any solvent which has a water solubility of less than 1 g/l, measured at 25° C. The term “water miscible solvent” has the opposite meaning to “water immiscible solvent”

As used herein, the term “polar aprotic solvent” refers to a polar solvent which lacks an acidic proton. Polar solvent is a synonym of water miscible solvent. In particular, the term “polar aprotic solvent” is used in this invention to cover any water miscible solvent which lacks an acidic proton and has a dielectric constant of 12 or higher, measured at 25° C.

The term “a” or “an” as used herein includes the singular and the plural, unless specifically stated otherwise. Therefore, the terms “a,” “an” or “at least one” can be used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, used of the term “about” herein specifically includes ±10% from the indicated values in the range. In addition, the endpoints of all ranges directed to the same component or property herein are inclusive of the endpoints, are independently combinable, and include all intermediate points and ranges. Similarly, the ranges and amounts for each element of the technology described herein can be used together with ranges or amounts for any of the other elements.

### Process for the Preparation of Biguanidine Compounds

The present disclosure relates to a process for the preparation of a biguanidine compound of formula (I) or an acid addition salt thereof:

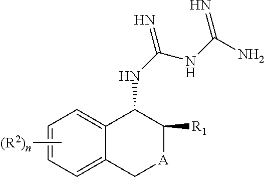

wherein R¹ and R² are each independently hydrogen or an optionally substituted C₁-C₄ alkyl group, wherein the optional substituents are selected from the group consisting of halogen, aliphatic, halo aliphatic, alicyclic, alkoxy, thioalkyl, cyano or nitro groups; A is —CH₂—, —O— or a direct bond; and n is 0, 1, 2 or 3;
which comprises the reaction of 1-cyanoguanidine with an amine of formula (II) or an acid addition salt thereof

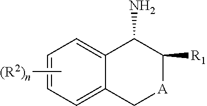

wherein R¹, R², A and n are as defined in formula (I), in a mixture of a polar aprotic solvent with a dielectric constant higher than 12.0, when measured at 25° C., and a water immiscible solvent.

This process provides an alternative to the existing processes for the preparation of biguanidine compounds, and particularly, it provides a solution for some of the problems in the state of the art of low yield, considerable amounts of waste to be handled, high temperature of the reaction or operationally complicated isolation. The mixture of a polar aprotic solvent with a dielectric constant higher than 12.0, when measured at 25° C., and a water immiscible solvent allows to obtain a the biguanidide compound of formula (I) in a high yield. Additionally, we have found that the mixture of a polar aprotic solvent with a dielectric constant higher than 12.0, when measured at 25° C., and a water immiscible solvent allows to lower the reaction temperature from 140° C.-148° C., and to obtain a good yield for the synthesis of the biguanidide compound of formula (I).

Prior art document EP3347342 A1 mentioned the reaction of amine compounds of formula (II) with cyanoguanidine in a polar aprotic solvent. According to that document, suitable polar aprotic solvents included anisole, n-hexyl acetate, dichlorobenzenes and mixtures thereof, where anisole was the preferred solvent. Anisole has a dielectric constant of 4.33, at 25° C.; n-hexyl acetate has a dielectric constant of 4.42; and o-, m-, p-dichlorobenzene have dielectric constants of 9.93, 5.00 and 2.41, respectively. All these solvents are water immiscible solvents and their water solubility is 0.4 g/l for hexyl acetate, 0.14 g/l for anisole, 0.08 g/l, 0.11 g/l and 0.049 g/l for o-, m-, p-dichlorobenzene, respectively. Although EP3347342 A1 discloses anisole, n-hexyl acetate and dichlorobenzenes as polar aprotic solvents, these solvents show a low polarity and they must be considered as non-polar solvents, and in fact, they are water immiscible solvents. The present invention differs from the prior art in that it contains a mixture of a polar aprotic solvent with a dielectric constant higher than 12.0, when measured at 25° C., and the water immiscible solvent.

The R¹ and R² may be each independently C₁-C₄ alkyl group and n may be 1 in the process of the invention. The R¹ and R² may be each independently C₁-C₄ alkyl group, n may be 1, and A may be a direct bond in the process of the invention. The R¹ and R² may be methyl groups and n may be 1 in the process of the invention. The R¹ and R² may be methyl groups, n may be 1, and A may be a direct bond in the process of the invention. The compound of formula (I) in the process of the invention may be (1R,2S)-1-(bisguanidino)-2,6-dimethylindane or (1R,2S)-1-(bisguanidino)-2,6-dimethylindane monohydrochloride and the compound of formula (II) may be (1R,2S)-1-amino-2,6-dimethylindane or (1R,2S)-1-amino-2,6-dimethylindane monohydrochloride.

The acid addition salt of the biguanidine of formula (I) or the amine of formula (II) may be the salt of an acid such as for example, hydrogen chloride, hydrogen bromide, hydrogen iodide, phosphoric acid, sulfuric acid, nitric acid, carbonic acid, mono- or bifunctional carboxylic acids and hydroxycarboxylic acids, such as acetic acid, oxalic acid, maleic acid, succinic acid, fumaric acid, tartaric acid, citric acid, salicylic acid, sorbic acid or lactic acid, and also sulfonic acids, such as methanesulfonic acid, p-toluenesulfonic acid or 1,5-naphthalenedisulfonic acid. The acid addition salt of the amine of formula (II) may be produced by the reaction of the amine of formula (II) with the acid by any conventional method known by the person skilled in the art.

The temperature of the reaction in the process of the invention may be in the range from 105° C. to 150° C., from 110° C. to 148° C., from 120° C. to 139° C., from 125° C. to 139° C. The temperature of the reaction in the process of the invention may be the temperature of reflux of the mixture of solvents. The mixture of the polar aprotic solvent with a dielectric constant higher than 12.0, when measured at 25° C., and the water immiscible solvent may have a boiling point of at least 105° C. The temperature of the reaction in the process of the invention may be the temperature of reflux of the mixture of the polar aprotic solvent with a dielectric constant higher than 12.0, when measured at 25° C., and the water immiscible solvent with a boiling point from 105° C. to 150° C., from 110° C. to 148° C., from 120° C. to 139° C., from 125° C. to 139° C. This reaction may be carried out at atmospheric pressure or a pressure higher than the atmospheric pressure.

The ratio by weight between the polar aprotic solvent with a dielectric constant higher than 12.0, when measured at 25° C., and the water immiscible solvent may range from 1:1 to 1:5, or from 1:1.5 to 1:3 in the mixture of solvents for the reaction of the process of the invention. The ratio by weight between the polar aprotic solvent with a dielectric constant higher than 12.0, when measured at 25° C., and the water immiscible solvent may range from 1:1 to 1:5, or from 1:1.5 to 1:3, and this mixture of solvents may have a boiling point of at least 105° C. The ratio by weight between the polar aprotic solvent with a dielectric constant higher than 12.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. The ratio by weight between the polar aprotic solvent with a dielectric constant higher than 12.0, when measured at 25° C., and the water immiscible solvent may range from 1:1 to 1:5, or from 1:1.5 to 1:3, this mixture of solvents may have a boiling point from 105° C. to 150° C., from 110° C. to 148° C., from 120° C. to 139° C., from 125° C. to 139° C., and the temperature of the reaction in the process of the invention may be the temperature of reflux of the mixture of solvents. This reaction may be carried out at atmospheric pressure or a pressure higher than the atmospheric pressure.

The polar aprotic solvent with a dielectric constant higher than 12.0, when measured at 25° C., may be added dropwise or it may be added at once to the mixture of 1-cyanoguanidine with the amine of formula (II) and the water immiscible solvent, or it may be added dropwise together with 1-cyanoguanidine to the mixture of the amine of formula (II) and the water immiscible solvent.

The polar aprotic solvent with a dielectric constant higher than 12.0, when measured at 25° C., may have a dielectric constant higher than 15.0, higher than 20.0, higher than 25.0.

The polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, v-butyrolactone, sulfolane, nitromethane, tetramethyl urea, propylene carbonate, hexamethylphosphoramide, butanone, methyl isobutyl ketone, acetone, pyridine or N,N-dimethylpropyleneurea. The polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone. The polar aprotic solvent with a dielectric constant higher than 20.0, when measured at 25° C., may be selected from the group consisting of N,N-dimethylformamide, N-methylpyrrolidone or dimethylsulfoxide. The polar aprotic solvent with a dielectric constant higher than 25.0, when measured at 25° C., may be N-methylpyrrolidone.

The polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea and the ratio by weight between the polar aprotic solvent with a dielectric constant higher than 12.0, when measured at 25° C., and the water immiscible solvent may range from 1:1 to 1:5, or from 1:1.5 to 1:3. The polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. The polar aprotic solvent with a dielectric constant higher than 20.0, when measured at 25° C., may be selected from the group consisting of N,N-dimethylformamide, N-methylpyrrolidone or dimethylsulfoxide, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. The polar aprotic solvent with a dielectric constant higher than 25.0, when measured at 25° C., may be N-methylpyrrolidone, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in the process of the invention may be the temperature of reflux of the mixture of solvents. This reaction may be carried out at atmospheric pressure or a pressure higher than the atmospheric pressure.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil. The water immiscible solvent may be selected from the group consisting of o-chlorobenzene, dichlorobenzene, m-dichlorobenzene, p-dichlorobenzene, anisole, hexyl acetate or white mineral oil. The water immiscible solvent may be chlorobenzene.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, and the polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, and the polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone. The water immiscible solvent may be selected from the group consisting of o-chlorobenzene, dichlorobenzene, m-dichlorobenzene, p-dichlorobenzene, anisole, hexyl acetate or white mineral oil, and the polar aprotic solvent with a dielectric constant higher than 20.0, when measured at 25° C., may be selected from the group consisting of N,N-dimethylformamide, N-methylpyrrolidone or dimethylsulfoxide. The water immiscible solvent may be chlorobenzene, and the polar aprotic solvent with a dielectric constant higher than 25.0, when measured at 25° C., may be N-methylpyrrolidone.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, and the ratio by weight between the polar aprotic solvent with a dielectric constant higher than 12.0, when measured at 25° C., and the water immiscible solvent may range from 1:1 to 1:5, or from 1:1.5 to 1:3. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. The water immiscible solvent may be selected from the group consisting of o-chlorobenzene, dichlorobenzene, m-dichlorobenzene, p-dichlorobenzene, anisole, hexyl acetate or white mineral oil, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. The water immiscible solvent may be chlorobenzene, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in the process of the invention may be the temperature of reflux of the mixture of solvents. This reaction may be carried out at atmospheric pressure or a pressure higher than the atmospheric pressure.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, the polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea, and the ratio by weight between the polar aprotic solvent with a dielectric constant higher than 12.0, when measured at 25° C., and the water immiscible solvent may range from 1:1 to 1:5, or from 1:1.5 to 1:3. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, the polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. The water immiscible solvent may be selected from the group consisting of o-chlorobenzene, dichlorobenzene, m-dichlorobenzene, p-dichlorobenzene, anisole, hexyl acetate or white mineral oil, the polar aprotic solvent with a dielectric constant higher than 20.0, when measured at 25° C., may be selected from the group consisting of N,N-dimethylformamide, N-methylpyrrolidone or dimethylsulfoxide, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. The water immiscible solvent may be chlorobenzene, the polar aprotic solvent with a dielectric constant higher than 25.0, when measured at 25° C., may be N-methylpyrrolidone, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in the process of the invention may be the temperature of reflux of the mixture of solvents. This reaction may be carried out at atmospheric pressure or a pressure higher than the atmospheric pressure.

The reaction in the process of the invention is carried out under a protective gas atmosphere.

### Process for the Preparation of Triazine Compounds

The present invention also relates to a process for the preparation of a triazine compound of formula (IV):

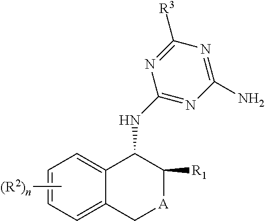

wherein R¹, R² and R³ are each independently hydrogen or an optionally substituted C₁-C₄ alkyl group, wherein the optional substituents are selected from the group consisting of halogen, aliphatic, halo aliphatic, alicyclic, alkoxy, thioalkyl, cyano or nitro groups; A is —CH₂—, —O— or a direct bond; and n is 0, 1, 2 or 3; which comprises
a) a first step of preparation of a biguanidine compound of formula (I) or an acid addition salt thereof:

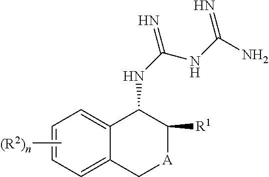

wherein R¹, R², A and n are as defined in formula (IV);
by reaction of 1-cyanoguanidine with an amine of formula (II) or an acid addition salt thereof

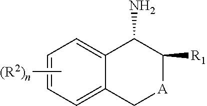

wherein R¹, R², A and n are as defined in formula (IV), in a mixture of a polar aprotic solvent with a dielectric constant higher than 12.0, when measured at 25° C., and a water immiscible solvent;
b) a base and a carboxylic acid derivative of formula (III) are added to the biguanidine compound of formula (I) or an acid addition salt thereof, obtained in step a)

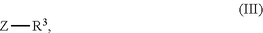

wherein R³ is as defined in formula (IV), and Z—R³ is selected from the group consisting of carboxylic acid esters, carboxylic orthoesters, carboxylic acid chlorides, carboxamides, nitriles, or carboxylic anhydrides.

This process provides an alternative to the existing processes for the preparation of triazine compounds of formula (IV). The mixture of a polar aprotic solvent with a dielectric constant higher than 12.0, when measured at 25° C., and a water immiscible solvent allows to obtain a the biguanidide compound of formula (I) in a high yield in step a) of the process. Additionally, we have found that the mixture of solvents in step a) allows to lower the reaction temperature from 140° C.-148° C., and to carry out the preparation of triazine compounds of formula (IV) in a one-pot synthesis.

The R¹ and R² may be each independently C₁-C₄ alkyl group, n may be 1, R³ may be halogen substituted C₁-C₄ alkyl group, and Z—R³ may be a carboxylic ester. The R¹ and R² may be each independently C₁-C₄ alkyl group, n may be 1, A may be a direct bond, R³ may be halogen substituted C₁-C₄ alkyl group, and Z—R³ may be a carboxylic ester. The R¹ and R² may be methyl groups, n may be 1 and R³ may be (R)-2-fluoropropionate group. The R¹ and R² may be methyl groups, n may be 1, A may be a direct bond, R³ may be (R)-2-fluoropropionate group and Z—R³ may be methyl-(R)-2-fluoropropionate. The compound of formula (IV) in the process of the invention may be N-[(1R,2S)-2,6-dimethyl-2,3-dihydro-1H-inden-yl]-6-[(1R)-1-fluoroethyl]-1,3-5-triazine-2,4-diamine, the compound of formula (I) may be (1R,2S)-1-(bisguanidino)-2,6-dimethylindane or (1R,2S)-1-(bisguanidino)-2,6-dimethylindane monohydrochloride and the compound of formula (II) may be (1R,2S)-1-amino-2,6-dimethylindane or (1R,2S)-1-amino-2,6-dimethylindane monohydrochloride.

The amount of the carboxylic acid derivative of formula (III) in step b) of the process of the invention may range from 1 to 5 moles, from 1 to 3 moles, from 1 to 2 moles by mole of the biguanidine compound of formula (I).

The acid addition salt of the biguanidine of formula (I) or the amine of formula (II) in step a) may be the salt of an acid such as for example, hydrogen chloride, hydrogen bromide, hydrogen iodide, phosphoric acid, sulfuric acid, nitric acid, carbonic acid, mono- or bifunctional carboxylic acids and hydroxycarboxylic acids, such as acetic acid, oxalic acid, maleic acid, succinic acid, fumaric acid, tartaric acid, citric acid, salicylic acid, sorbic acid or lactic acid, and also sulfonic acids, such as methanesulfonic acid, p-toluenesulfonic acid or 1,5-naphthalenedisulfonic acid. The acid addition salt of the amine of formula (II) may be produced by the reaction of the amine of formula (II) with the acid by any conventional method known by the person skilled in the art.

The base in step b) of the process of the invention may be selected from the group consisting of an alkali metal, alkaline earth metal or ammonium hydroxide, hydride, alkoxide, carbonate, bicarbonate, phosphate, hydrogen- or dihydrogenphosphate, or a tertiary or aromatic amine or mixtures thereof. The base in step b) of the process of the invention may be selected from the group consisting of an alkali metal or alkaline earth metal hydroxide, alkoxide, carbonate, bicarbonate, or mixtures thereof. The base in step b) of the process of the invention may be selected from the group consisting of an alkali metal hydroxide, methoxide, ethoxide, propoxide, butoxide, carbonate or mixtures thereof.

The amount of the base in step b) of the process of the invention may range from 1 to 5 moles, from 1 to 4 moles, from 1 to 3 moles by mole of the biguanidine compound of formula (I).

The amount of the base in step b) of the process of the invention may range from 1 to 5 moles by mole of the biguanidine compound of formula (I) and the base may be selected from the group consisting of an alkali metal, alkaline earth metal or ammonium hydroxide, hydride, alkoxide, carbonate, bicarbonate, phosphate, hydrogen- or dihydrogenphosphate, or a tertiary or aromatic amine or mixtures thereof. The amount of the base in step b) of the process of the invention may range from 1 to 4 moles by mole of the biguanidine compound of formula (I) and the base may be selected from the group consisting of an alkali metal or alkaline earth metal hydroxide, alkoxide, carbonate, bicarbonate, or mixtures thereof. The amount of the base in step b) of the process of the invention may range from 1 to 3 moles by mole of the biguanidine compound of formula (I) and the base may be selected from the group consisting of an alkali metal hydroxide, methoxide, ethoxide, propoxide, butoxide, carbonate or mixtures thereof.

The solvent of the reaction in step b) may be any organic solvent. The solvent of the reaction in step b) may be selected from the group consisting of optionally halogen substituted aromatic hydrocarbons, optionally halogen substituted aliphatic hydrocarbons, nitrogen heterocyclic compounds, optionally alkyl substituted cyclic ethers, aliphatic ethers, ethers of aromatic hydrocarbons, nitriles, ketones, esters, amides, alcohols or glycols. The solvent of the reaction in step b) may be selected from the group consisting of toluene, benzene, ethylbenzene, xylenes, cumene, cymenes, mesitylene, biphenyl, chlorobenzene, bromobenzene, o-dichlorobenzene, m-dichlorobenzene, p-dichlorobenzene, chloromethane, dichloromethane, trichloromethane, tetrachloromethane, dichloroethane, trichloroethane, trichloroethylene, tetrachloroethylene, pentane, hexane, heptane, cyclohexane, decalin, quinoline, isoquinoline, quinoxaline, phthalazine, quinazoline, cinnoline, pyridine, pyridazine, pyrimidine, pyrazine, triazines, 1,4-dioxane, tetrahydropyran, methyl tetrahydropyran, tetrahydrofuran, methyl tetrahydrofuran, dibutylether, tert-butyl methyl ether, anisole, acetonitrile, benzonitrile, acetone, butanone, ethyl acetate, n-butyl acetate, hexyl acetate, dimethylformamide or dimethylacetamide, ethanol, methanol, butanol, tert-butanol, propanol, isopropyl alcohol, isoamyl alcohol, phenol, ethylene glycol, propylene glycol, diethylene glycol or dimethoxyethane. The solvent of the reaction in step b) may be selected from the group consisting of toluene, 1,4-dioxane, tetrahydropyran, methyl tetrahydropyran, tetrahydrofuran, methyl tetrahydrofuran, anisole, acetonitrile, dimethylformamide, dimethylacetamide, ethanol, methanol or butanol. The solvent of the reaction in step b) may be ethanol or methanol.

The temperature of the reaction in step a) of the process of the invention may be in the range from 105° C. to 150° C., from 110° C. to 148° C., from 120° C. to 139° C., from 125° C. to 139° C. The temperature of the reaction in step a) of the process of the invention may be the temperature of reflux of the mixture of solvents. The mixture of the polar aprotic solvent with a dielectric constant higher than 12.0, when measured at 25° C., and the water immiscible solvent in step a) may have a boiling point of at least 105° C. The temperature of the reaction in step a) of the process of the invention may be the temperature of reflux of the mixture of the polar aprotic solvent with a dielectric constant higher than 12.0, when measured at 25° C., and the water immiscible solvent with a boiling point from 105° C. to 150° C., from 110° C. to 148° C., from 120° C. to 139° C., from 125° C. to 139° C. This reaction of step a) may be carried out at atmospheric pressure or a pressure higher than the atmospheric pressure.

The ratio by weight between the polar aprotic solvent with a dielectric constant higher than 12.0, when measured at 25° C., and the water immiscible solvent may range from 1:1 to 1:5, or from 1:1.5 to 1:3 in the mixture of solvents for the reaction in step a) of the process of the invention. The ratio by weight between the polar aprotic solvent with a dielectric constant higher than 12.0, when measured at 25° C., and the water immiscible solvent may range from 1:1 to 1:5, or from 1:1.5 to 1:3, and this mixture of solvents in step a) may have a boiling point of at least 105° C. The ratio by weight between the polar aprotic solvent with a dielectric constant higher than 12.0, when measured at 25° C., and the water immiscible solvent may range from 1:1 to 1:5, or from 1:1.5 to 1:3, and this mixture of solvents in step a) may have a boiling point from 105° C. to 150° C., from 110° C. to 148° C., from 120° C. to 139° C., from 125° C. to 139° C. The ratio by weight between the polar aprotic solvent with a dielectric constant higher than 12.0, when measured at 25° C., and the water immiscible solvent may range from 1:1 to 1:5, or from 1:1.5 to 1:3, this mixture of solvents in step a) may have a boiling point from 105° C. to 150° C., from 110° C. to 148° C., from 120° C. to 139° C., from 125° C. to 139° C., and the temperature of the reaction in step a) of the process of the invention may be the temperature of reflux of the mixture of solvents. This reaction of step a) may be carried out at atmospheric pressure or a pressure higher than the atmospheric pressure.

The polar aprotic solvent with a dielectric constant higher than 12.0, when measured at 25° C., may be added dropwise or it may be added at once to the mixture of 1-cyanoguanidine with the amine of formula (II) and the water immiscible solvent in step a), or it may be added dropwise together with 1-cyanoguanidine to the mixture of the amine of formula (II) and the water immiscible solvent in step a) of the process of the invention.

The polar aprotic solvent with a dielectric constant higher than 12.0, when measured at 25° C., in step a) may have a dielectric constant higher than 15.0, higher than 20.0, higher than 25.0.

The polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea in step a) of the process of the invention. The polar aprotic solvent with a dielectric constant higher than 15.0, when measured at 25° C., in step a) may be selected from the group consisting of N,N-dimethylformamide, dimethylacetamide, N-methylpyrrolidone, dimethylsulfoxide, acetonitrile, benzonitrile, butanone, methyl isobutyl ketone or acetone. The polar aprotic solvent with a dielectric constant higher than 20.0, when measured at 25° C., in step a) may be selected from the group consisting of N, N-dimethylformamide, N-methylpyrrolidone or dimethylsulfoxide. The polar aprotic solvent with a dielectric constant higher than 25.0, when measured at 25° C., in step a) may be N-methylpyrrolidone.

The polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea and the ratio by weight between the polar aprotic solvent with a dielectric constant higher than 12.0, when measured at 25° C., and the water immiscible solvent may range from 1:1 to 1:5, or from 1:1.5 to 1:3 in step a) of the process of the invention. The polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. in step a) of the process of the invention. The polar aprotic solvent with a dielectric constant higher than 20.0, when measured at 25° C., may be selected from the group consisting of N,N-dimethylformamide, N-methylpyrrolidone or dimethylsulfoxide, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. in step a) of the process of the invention. The polar aprotic solvent with a dielectric constant higher than 25.0, when measured at 25° C., may be N-methylpyrrolidone, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in step a) of the process of the invention may be the temperature of reflux of the mixture of solvents. This reaction of step a) may be carried out at atmospheric pressure or a pressure higher than the atmospheric pressure.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether in step a) of the process of the invention. The water immiscible solvent in step a) may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil. The water immiscible solvent in step a) may be selected from the group consisting of o-chlorobenzene, dichlorobenzene, m-dichlorobenzene, p-dichlorobenzene, anisole, hexyl acetate or white mineral oil. The water immiscible solvent in step a) may be chlorobenzene.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, and the polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea in step a) of the process of the invention. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, and the polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone in step a) of the process of the invention. The water immiscible solvent may be selected from the group consisting of o-chlorobenzene, dichlorobenzene, m-dichlorobenzene, p-dichlorobenzene, anisole, hexyl acetate or white mineral oil, and the polar aprotic solvent with a dielectric constant higher than 20.0, when measured at 25° C., may be selected from the group consisting of N,N-dimethylformamide, N-methylpyrrolidone or dimethylsulfoxide in step a) of the process of the invention. The water immiscible solvent may be chlorobenzene, and the polar aprotic solvent with a dielectric constant higher than 25.0, when measured at 25° C., may be N-methylpyrrolidone in step a) of the process of the invention.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, and the ratio by weight between the polar aprotic solvent with a dielectric constant higher than 12.0, when measured at 25° C., and the water immiscible solvent may range from 1:1 to 1:5, or from 1:1.5 to 1:3 in step a) of the process of the invention. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. in step a) of the process of the invention. The water immiscible solvent may be selected from the group consisting of o-chlorobenzene, dichlorobenzene, m-dichlorobenzene, p-dichlorobenzene, anisole, hexyl acetate or white mineral oil, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. in step a) of the process of the invention. The water immiscible solvent may be chlorobenzene, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in step a) of the process of the invention may be the temperature of reflux of the mixture of solvents. This reaction of step a) may be carried out at atmospheric pressure or a pressure higher than the atmospheric pressure.

The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether, the polar aprotic solvent with a dielectric constant higher than 12.0, when measured at 25° C., may be selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea, and the ratio by weight between the polar aprotic solvent with a dielectric constant higher than 12.0, when measured at 25° C., and the water immiscible solvent may range from 1:1 to 1:5, or from 1:1.5 to 1:3 in step a) of the process of the invention. The water immiscible solvent may be selected from the group consisting of chlorobenzene, bromobenzene, o-dichlorobenzene, m-dichlorobenzene, p-dichlorobenzene, anisole, toluene, benzene, ethylbenzene, xylenes, cymenes, n-butyl acetate, hexyl acetate, white mineral oil, the polar aprotic solvent with a dielectric constant higher than 15.0, when measured at 25° C., may be selected from the group consisting of N,N-dimethylformamide, dimethylacetamide, N-methylpyrrolidone, dimethylsulfoxide, acetonitrile, benzonitrile, butanone, methyl isobutyl ketone or acetone, the ratio by weight between the polar aprotic solvent with a dielectric constant higher than 15.0, when measured at 25° C., and the water immiscible solvent may range from 1:1 to 1:5, or from 1:1.5 to 1:3, and this mixture of solvents may have a boiling point of at least 105° C. in step a) of the process of the invention. The water immiscible solvent may be selected from the group consisting of o-chlorobenzene, dichlorobenzene, m-dichlorobenzene, p-dichlorobenzene, anisole, hexyl acetate or white mineral oil, the polar aprotic solvent with a dielectric constant higher than 20.0, when measured at 25° C., may be selected from the group consisting of N,N-dimethylformamide, N-methylpyrrolidone or dimethylsulfoxide, the ratio by weight between the polar aprotic solvent with a dielectric constant higher than 20.0, when measured at 25° C., and the water immiscible solvent may range from 1:1 to 1:5, or from 1:1.5 to 1:3, and this mixture of solvents may have a boiling point from 105° C. to 150° C., from 110° C. to 148° C., from 120° C. to 139° C., from 125° C. to 139° C. in step a) of the process of the invention. The water immiscible solvent may be chlorobenzene, the polar aprotic solvent with a dielectric constant higher than 25.0, when measured at 25° C., may be N-methylpyrrolidone, the ratio by weight between the polar aprotic solvent with a dielectric constant higher than 25.0, when measured at 25° C., and the water immiscible solvent may range from 1:1 to 1:5, or from 1:1.5 to 1:3, this mixture of solvents may have a boiling point from 120° C. to 139° C., from 125° C. to 139° C., and the temperature of the reaction in step a) of the process of the invention may be the temperature of reflux of the mixture of solvents. This reaction in step a) may be carried out at atmospheric pressure or a pressure higher than the atmospheric pressure.

The process for the preparation of the triazine compound of formula (IV) may be carried out in one-pot synthesis without isolation of the biguanidine compound of formula (I) or an acid addition salt thereof, or it may be performed in 2 separate steps with isolation of the biguanidine compound of formula (I) or an acid addition salt thereof after step a).

In one embodiment, the biguanidine compound of formula (I) or an acid addition salt thereof may be isolated by any conventional method known by the person skilled in the art, including but not limited to filtration, washing the reaction product with a solvent or mixture of solvents in order to dissolve the impurities of the biguanidine compound of formula (I) or an acid addition salt thereof, or by crystallization of the biguanidine compound of formula (I) or an acid addition salt thereof.

In other embodiment, the biguanidine compound of formula (I) or an acid addition salt thereof is not isolated, then, the preparation of the triazine compound of formula (IV) is carried out in a one-pot synthesis, and the water immiscible solvent from step a) is removed before the reaction of step b) of the process of the invention. The water immiscible solvent may be removed by any conventional method known by the person skilled in the art, including but not limited to distillation, or distillation under vacuum.

The temperature of step b) of the process of the invention may be from room temperature to the reflux temperature of the solvent in step b).

The steps a) and b) of the process of the invention are carried out under a protective gas atmosphere.

The following examples are included for illustrative purposes only and should not be construed as limitations on the invention claimed herein.

### EXAMPLES

### Example 1: Synthesis of N-[(1R,2S)-2,6-dimethyl-2,3-dihydro-1H-inden-yl]-6-[(1R)-1-fluoroethyl]-1,3-5-triazine-2,4-diamine with monochlorobenzene and N-methylpyrrolidone as solvents

(1R,2S)-2,6-dimethyl-2,3-dihydro-1H-inden-1-amine HCl salt (15 gr, 0.077 mol, 1 equiv.) is dissolved in 75 ml of monochlorobenzene and the mixture is heated to 130° C. under a protective gas atmosphere. Next, 1-cyanoguanidine (13 gr, 0.154 mol, 2 equiv.), which is previously dissolved in 45 ml of N-methylpyrrolidone, is added dropwise. Then, the reaction mixture is stirred for 8 h at 130° C. Upon completion, the mixture is cooled to 50° C., and then monochlorobenzene is distilled off under vacuum. Afterwards, the reaction mixture is cooled to room temperature under a protective gas atmosphere and then 30 ml of MeOH is added to the flask, followed by dropwise addition of NaOMe 30% solution in MeOH (34.7 gr, 0.193 mol, 2.5 equiv.) and methyl-(R)-2-fluoropropionate (16 gr, 0.154 mol, 2 equiv.). The mixture is stirred for additional 4 h. At the end of the reaction, water is added dropwise and stirred for another 0.5 h. Afterwards, the mixture is filtered, and the cake is washed with water. Finally, the material is dried in oven at 70° C. under vacuum. Chemical yield obtained is 84%.

### Comparative example 2: Synthesis of N-[(1R,2S)-2,6-dimethyl-2,3-dihydro-1H-inden-yl]-6-[(1R)-1-fluoroethyl]-1,3-5-triazine-2,4-diamine with anisole as solvent

(1R,2S)-2,6-dimethyl-2,3-dihydro-1H-inden-1-amine HCl salt (10 gr, 0.05 mol, 1 equiv.) is dissolved in 100 ml of anisole and the mixture is heated to 140° C. under a protective gas atmosphere. Next, 1-cyanoguanidine (6.4 gr, 0.076 mol, 1.5 equiv.) is added portion wise, followed by the addition of Tetrabutyl ammonium Bromide (0.8 gr, 0.0025 mol, 0.05 eq). Then, the reaction mixture is stirred for 3.5 h at 140° C. Upon completion, the mixture is cooled to room temperature and then, anisole is distilled off under vacuum. Afterwards, the reaction mixture is cooled to room temperature under a protective gas atmosphere and then, 50 ml of MeOH are added to the flask, followed by dropwise addition of NaOMe 30% solution in MeOH (22.5 gr, 0.125 mol, 2.5 equiv.) and methyl-(R)-2-fluoropropionate (10.6 gr, 0.1 mol, 2 equiv.) The mixture is stirred for additional 4 h. At the end of the reaction, water is added dropwise and stirred for another 0.5 h. Afterwards, the mixture is filtered, and the cake is washed with water. Finally, the material is dried in oven at 70° C. under vacuum. Chemical yield obtained was 67%.

### Comparative Example 3: Synthesis of N-[(1R,2S)-2,6-dimethyl-2,3-dihydro-1H-inden-yl]-6-[(1R)-1-fluoroethyl]-1,3-5-triazine-2,4-diamine with anisole as solvent and K₂CO₃ as base

(1R,2S)-2,6-dimethyl-2,3-dihydro-1H-inden-1-amine HCl salt (7 gr, 0.035 mol, 1 equiv.) is dissolved in 70 ml of anisole and the mixture is heated to 145° C. under a protective gas atmosphere. Next, 1-cyanoguanidine (5.9 gr, 0.07 mol, 2 equiv.) is added portion wise. Then, the reaction mixture is stirred for 8.5 h at 145° C. Upon completion, the mixture is cooled to 90° C. Afterwards, K₂CO₃ (19.3 gr, 0.14 mol, 4 eq) is added at once, followed by the addition of methyl-(R)-2-fluoropropionate (7.8 gr, 0.07 mol, 2.1 equiv.) under a protective gas atmosphere. The reaction mixture is stirred at 90° C. for 16 h. At the end of the reaction, salts are filtrated and washed with hot anisole. The chemical yield obtained in the combined filtrates is 61%.

1. A process for the preparation of a biguanidine compound of formula (I) or an acid addition salt thereof: 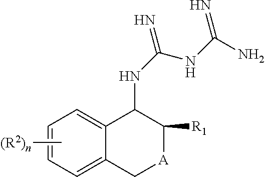
wherein R¹ and R² are each independently hydrogen or an optionally substituted C₁-C₄ alkyl group, wherein the optional substituents are selected from the group consisting of halogen, aliphatic, halo aliphatic, alicyclic, alkoxy, thioalkyl, cyano or nitro groups; A is —CH₂—, —O— or a direct bond; and n is 0, 1, 2 or 3;
which comprises the reaction of 1-cyanoguanidine with an amine of formula (II) or an acid addition salt thereof 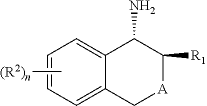
wherein R¹, R², A and n are as defined in formula (I), in a mixture of a polar aprotic solvent with a dielectric constant higher than 12.0, when measured at 25° C., and a water immiscible solvent.

2. The process according to claim 1, wherein R¹ and R² are each independently C₁-C₄ alkyl group and n is 1.

3. The process according to any of previous claims, wherein the compound of formula (I) is (1R,2S)-1-(bisguanidino)-2,6-dimethylindane or (1R,2S)-1-(bisguanidino)-2,6-dimethylindane monohydrochloride and the compound of formula (II) is (1R,2S)-1-amino-2,6-dimethylindane or (1R,2S)-1-amino-2,6-dimethylindane monohydrochloride.

4. The process according to any of previous claims, wherein the temperature of the reaction is in the range from 105° C. to 150° C.

5. The process according to any of previous claims, wherein the dielectric constant of the polar aprotic solvent is higher than 15.0, when measured at 25° C.

6. The process according to any of previous claims, wherein the ratio by weight between the polar aprotic solvent with a dielectric constant higher than 12.0, when measured at 25° C., and the water immiscible solvent ranges from 1:1 to 1:5.

7. The process according to any of previous claims, wherein the polar aprotic solvent with a dielectric constant higher than 12.0, when measured at 25° C., is selected from the group consisting of N,N-dimethylformamide, dimethylacetamide, N-methylpyrrolidone, dimethylsulfoxide, acetonitrile, benzonitrile, nitromethane, nitrobenzene, γ-butyrolactone, sulfolane, nitromethane, tetramethyl urea, propylene carbonate, hexamethylphosphoramide, butanone, methyl isobutyl ketone, acetone, pyridine or N,N-dimethylpropyleneurea.

8. The process according to any of previous claims, wherein the water immiscible solvent is selected from the group consisting of chlorobenzene, bromobenzene, o-dichlorobenzene, m-dichlorobenzene, p-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene or dibutylether.

9. A process for the preparation of a triazine compound of formula (IV): 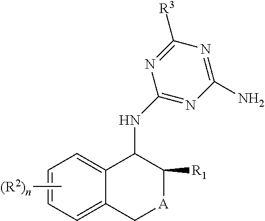
wherein R¹, R² and R³ are each independently hydrogen or an optionally substituted C₁-C₄ alkyl group, wherein the optional substituents are selected from the group consisting of halogen, aliphatic, halo aliphatic, alicyclic, alkoxy, thioalkyl, cyano or nitro groups; A is —CH₂—, —O— or a direct bond; and n is 0, 1, 2 or 3; which comprises
a) a first step of preparation of a biguanidine compound of formula (I) or an acid addition salt thereof according to any of previous claims;
b) a base and a carboxylic acid derivative of formula (III) are added to the biguanidine compound of formula (I) or an acid addition salt thereof, obtained in step a) 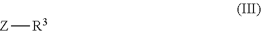
wherein R³ is as defined in formula (IV), and Z—R³ is selected from the group consisting of carboxylic acid esters, carboxylic orthoesters, carboxylic acid chlorides, carboxamides, nitriles, or carboxylic anhydrides.

10. The process according to claim 9, wherein R¹ and R² are each independently C₁-C₄ alkyl group, n is 1, R³ is halogen substituted C₁-C₄ alkyl group, and Z—R³ is a carboxylic ester.

11. The process according to any of claims 9 to 10, wherein the compound of formula (IV) is N-[(1R,2S)-2,6-dimethyl-2,3-dihydro-1H-inden-yl]-6-[(1R)-1-fluoroethyl]-1,3-5-triazine-2,4-diamine, the compound of formula (I) is (1R,2S)-1-(bisguanidino)-2,6-dimethylindane or (1R,2S)-1-(bisguanidino)-2,6-dimethylindane monohydrochloride and the compound of formula (II) is (1R,2S)-1-amino-2,6-dimethylindane or (1R,2S)-1-amino-2,6-dimethylindane monohydrochloride.

12. The process according to any of claims 9 to 11, wherein the temperature of the reaction in step a) is in the range from 105° C. to 150° C.

13. The process according to any of claims 9 to 12, wherein the ratio by weight between the polar aprotic solvent with a dielectric constant higher than 12.0, when measured at 25° C., and the water immiscible solvent in step a) ranges from 1:1 to 1:5.

14. The process according to any of claims 9 to 13, wherein the polar aprotic solvent with a dielectric constant higher than 12.0, when measured at 25° C., in step a) is selected from the group consisting of N,N-dimethylformamide, dimethylacetamide, N-methylpyrrolidone, dimethylsulfoxide, acetonitrile, nitromethane, γ-butyrolactone, sulfolane, nitromethane, tetramethyl urea, propylene carbonate, hexamethylphosphoramide, butanone, acetone, pyridine or N,N-dimethylpropyleneurea.

15. The process according to any of claims 9 to 14, wherein the water immiscible solvent in step a) is selected from the group consisting of chlorobenzene, bromobenzene, 1,2-dichlorobenzene, 1,3-dichlorobenzene, anisole, toluene, benzene, ethylbenzene, xylenes, cumene, cymenes, mesitylene, biphenyl, decalin, n-butyl acetate, hexyl acetate, white mineral oil, tetrachloroethylene, dibutylether.
